(19) United States
(12) Patent Application Publication
(10) Pub. No.: US 2012/0271029 A1
(43) Pub. Date: Oct. 25, 2012

(54) PROCESS FOR PREPARING NEOPENTYL GLYCOL

(75) Inventors: Helmut Kronemayer, Heidelberg (DE); Michael Steiniger, Neustadt (DE); Eva Kretzschmar, Mannheim (DE); Norbert Asprion, Ludwigshafen (DE); Marcus Bechtel, Heidelberg (DE)

(73) Assignee: BASF SE, Ludwigshafen (DE)

(21) Appl. No.: 13/446,631

(22) Filed: Apr. 13, 2012

Related U.S. Application Data
(60) Provisional application No. 61/476,793, filed on Apr. 19, 2011.

(51) Int. Cl. C08G 63/00; C07C 29/74; C07C 45/78

(52) U.S. Cl. 528/271; 568/464; 568/854

(57) ABSTRACT

The present invention relates to a process for preparing hydroxypivalaldehyde (HPA), which comprises in a first stage reacting isobutyraldehyde with formaldehyde in the presence of a tertiary amine and in a second stage introducing the reaction output obtained from the first stage into a stripping column. The present application further relates to a process for preparing neopentyl glycol (NPG) by hydrogenating the hydroxypivalaldehyde prepared in accordance with the invention and the further conversion of the NPG thus obtained to polyester resins, unsaturated polyester resins, lubricants or plasticizers.

The present application incorporates provisional U.S. application No. 61/476,793, filed Apr. 19, 2012, by reference.

The present invention relates to a process for preparing hydroxypivalaldehyde (HPA) by reacting isobutyraldehyde (IBA) with formaldehyde (FA). The present application further relates to a process for preparing neopentyl glycol (NPG) by hydrogenating the hydroxypivalaldehyde prepared in accordance with the invention, and to the further conversion of the NPG thus obtained to polyester resins, unsaturated polyester resins, lubricants or plasticizers.

Neopentyl glycol is used as a raw material for the production of saturated polyester resins, for powder coating materials and for glass fiber reinforced polymers.

Neopentyl glycol is generally prepared in a two-stage process, in which isobutyraldehyde (IBA) is first reacted with formaldehyde (FA) in an aldol addition to give hydroxypivalaldehyde (HPA), which can be hydrogenated in a second process stage directly to neopentyl glycol (NPG).

In the first process stage (aldol reaction), isobutyraldehyde is generally reacted with formaldehyde in an aldol reaction in the presence of tertiary amines as a catalyst. The output from the aldol reaction typically comprises HPA and unconverted starting compounds, such as formaldehyde, IBA, and also the tertiary amine catalyst used and water. The output typically also comprises impurities and by-products from the aldol reaction, such as formic acid which can form from formaldehyde by Cannizzaro or Tishchenko reaction, and formate salts of the amine catalysts used, such as trimethylammonium formate.

After the aldolization, unconverted aldehydes and a portion of the amine base are generally removed by distillation and recycled into the aldol reaction.

For instance, WO 97/17313 describes a process for preparing hydroxyalkanes by reacting the corresponding aldehydes with formaldehyde in an aqueous solution in the presence of tertiary amine in a reactor cascade composed of several stirred tanks connected in series. The resulting reaction mixture is generally purified in several columns.

WO 98/28253 likewise discloses a multistage process for preparing hydroxyalkanals, wherein in the first stage the corresponding starting aldehyde is reacted with FA in the presence of trialkylamines, and in a second stage the reaction mixture from stage 1 is separated by distillation into bottoms which comprise the reaction products, and a top product composed of lower-boiling components, which can be recycled into the first stage. In a third stage the bottoms from the second stage are introduced into a postreaction or distillation stage, in which the incompletely methylolated compounds are converted to the corresponding methylene compounds and distilled off overhead. The bottom product obtained in the distillation is sent to a hydrogenation.

U.S. Pat. No. 4,036,888, GB 1219162 and JP-A 3193738 also describe two-stage processes for preparing NPG, in which IBA is first reacted under base catalysis with formaldehyde to give HPA, which is then catalytically hydrogenated to NPG.

An overview of a two-stage process for preparing NPG can be found, for example, in WO-A1-2010079187.

It was an object of the present invention to provide a process for preparing HPA which can be introduced directly into a downstream hydrogenation stage in which HPA is converted to NPG, with substantial avoidance of thermal decomposition of the HPA. There should be provision of an aldolization output which leads in a downstream hydrogenation stage to higher NPG yields and to a lower level of unwanted side reactions. It was a further object of the present invention to reduce the number of formaldehyde equivalents used in the aldolization reaction, in order to save raw material costs. It was a further object of the present invention to provide an aldolization output with a low content of unconverted isobutyraldehyde. In addition, there was to be provision of a process in which the formation of HPN (ester of hydroxypivalic acid and NPG (=HPN)) can be reduced. The process according to the invention was also to enable production of a hydrogenation feed with a pH of 7.5 to 8.5 without addition of further pH regulators.

The object of the present invention is achieved by a

process for preparing hydroxypivalaldehyde (HPA), which comprises in a first stage reacting isobutyraldehyde (IBA) with formaldehyde (FA) in the presence of a tertiary amine and in a second stage introducing the reaction output obtained from the first stage into a stripping column, with introduction of a stripping gas into the stripping column.

According to the invention, in a first reaction stage isobutyraldehyde is reacted with formaldehyde in the presence of a tertiary amine.

Formaldehyde is generally used in the process as an aqueous formaldehyde solution. Industrially available formaldehyde is sold typically in aqueous solution in concentrations of 30, 37 and 49% by weight. However, it is also possible to use formaldehyde solutions of up to 60% by weight in the process.

Industrial formaldehyde generally comprises formic acid as a result of the preparation. The degradation products of formic acid can reduce the service life of the hydrogenation catalyst in the downstream hydrogenation stage, which can result in a decrease in the yield of NPG. In a particular embodiment, formaldehyde which has a formic acid content of 150 ppm or less is used. Such formaldehyde can be obtained, as described in the application WO 2008107333, by treatment of formaldehyde or of an aqueous formaldehyde solution with basic ion exchangers. Useful anion exchangers include ion exchangers which are known per se, are strongly basic, weakly basic or moderately basic, and are in gel form or macroporous form. These are, for example, anion exchangers of the polystyrene resin structure crosslinked with divinylbenzene, having tertiary amino groups as functional groups. Also useful are ion exchangers based on acrylic acid or methacrylic acid crosslinked with divinylbenzene, or resins prepared by condensation of formaldehyde and phenol. Specific useful examples include the commercial products Ambersep® 900, Amberlyst® and Amberlite® from Rohm and Haas, Philadelphia, USA, and Lewatit® from Lanxess, Leverkusen.

Isobutyraldehyde is also used in the process according to the invention.

The preparation of isobutyraldehyde is described, for example, in the chapter “Butanals” (Ullmann's Encyclopedia of Industrial Chemistry, Published Online: 15 Sep. 2000, DOI: 10.1002/14356007.a04_{—}447). It can be prepared, for example, by hydroformylation of propylene.

The purity of the isobutyraldehyde used is preferably more than 95% by weight, more preferably more than 97% by weight and more preferably more than 99% by weight.

The tertiary amines used may be amines as described, for example, in DE-A 28 13 201 and DE-A 27 02 582. Particular preference is given to tri-n-alkylamines, especially triethylamine, tri-n-propylamine, tri-n-butylamine and trimethylamine.

Very particular preference is given to trimethylamine (“TMA”), triethylamine (“TEA”) and tri-n-propylamine (“TPA”), since these compounds generally have a lower boiling point than NPG and hence distillative removal from the reaction mixture is facilitated. Particular preference is given to using trimethylamine (“TMA”) as the tertiary amine in the reaction.

The aldol reaction (first stage) can be performed with or without addition of organic solvents or solubilizers. The use of solvents which form suitable low-boiling azeotropic mixtures with the low-boiling compounds in the individual distillations of the process according to the invention can possibly lower the energy expenditure in these distillations and/or facilitate the distillative removal of the low boilers from the high-boiling compounds.

Suitable solvents are, for example, cyclic and acyclic ethers, such as THF, dioxane, methyl tert-butyl ether, or alcohols such as methanol, ethanol or 2-ethylhexanol.

In the aldol reaction, the molar ratio of isobutyraldehyde added in fresh form in each case to the amount of formaldehyde added is appropriately in the range from 1:1 to 1:5, preferably in the range from 1:1.01 to 1:3.5 and more preferably in the range from 1:1.02 to 1:1.5 and especially preferably in the range from 1:1.03 to 1:1.1.

The amount of tertiary amine catalyst added in the aldol reaction is, in relation to the isobutyraldehyde added, generally 0.001 to 0.2, preferably 0.01 to 0.07, equivalent, which means that the amine is typically used in catalytic amounts.

The aldol reaction is performed generally at a temperature of 5 to 100° C., preferably of 15 to 80° C.

The reactions described for the aldol reaction can be performed at a pressure of generally 1 to 30 bar, preferably 1 to 15 bar, more preferably 1 to 5 bar, appropriately under the autogenous pressure of the reaction system in question.

The aldol reaction can be performed batchwise or continuously.

The aldol reaction is preferably performed in a continuous stirred tank reactor or a continuous stirred tank cascade. To adjust the residence time, a portion of the reaction output from one stirred tank can be recycled into the particular stirred tank reactor.

The overall residence time of the first stage (aldolization) is preferably 0.25 to 12 hours, more preferably 0.5 to 8 hours and especially preferably 1 to 3 hours. In a preferred embodiment, the residence time in the individual reactors of a reactor cascade is preferably selected such that the overall residence time is divided equally between the individual reactors.

The output from the aldol reaction comprises typically unconverted starting compounds, such as formaldehyde, alkanals, and also the tertiary amine catalyst used and possibly water.

The output additionally comprises hydroxypivalaldehyde (HPA). The output typically also comprises impurities and by-products from the aldol reaction, such as formic acid, which can form as a result of Cannizzaro or Tishchenko reaction from formaldehyde, such as HPN, and formate salts of the amine catalysts used, such as trimethylammonium formate.

The output from the aldolization comprises preferably 40 to 80% by weight of HPA and more preferably 50 to 70% by weight of HPA.

In a preferred embodiment, the output from the aldolization has the following composition:

HPA: 40 to 80% by weight;

water: 10 to 50% by weight;

IBA: 0 to 20% by weight;

FA: 0 to 10% by weight;

tert. amine: 0 to 10% by weight.

In a further preferred embodiment, the output from the aldolization has the following composition:

HPA: 50 to 70% by weight;

water: 15 to 40% by weight;

IBA: 1 to 10% by weight;

FA: 0.5 to 5% by weight;

tert. amine: 0.5 to 5% by weight.

According to the invention, the output from the first stage (aldolization) in a second reaction stage is introduced into a stripping column, into which a stripping gas is also introduced.

The stripping column is preferably an apparatus for separation of low-boiling and high-boiling components, the HPA-comprising reaction output from the first stage preferably being fed in in the upper region of the column, and an HPA-containing reaction output being drawn off in the lower region of the columns, for example in the column bottom.

At the top of the column or in the upper region of the columns, for example in a spatial area of 10 to 100%, preferably 50 to 100%, of theoretical plates, a gaseous stream is generally drawn off, which comprises, as significant components in addition to the stripping gas, unconverted isobutyraldehyde, tertiary amine, methanol and possibly water and formaldehyde.

According to the separating performance of the column used, exact operating conditions of the stripping column can be determined in a routine manner by the person skilled in the art on the basis of the known vapor pressures and evaporation equilibria of the components present in the output from the first stage by conventional calculation methods.

The second reaction stage is preferably performed in a stripping column which has internals for increasing the separating performance.

The second reaction stage is more preferably performed in a tray column. In a tray column, intermediate trays on which the mass transfer takes place are present in the interior of the column. Examples of different tray types are sieve trays, tunnel-cap trays, dual-flow trays, bubble-cap trays, valve trays or Streuber trays. The bubble-cap trays used may also be modified bubble-cap trays with deflecting plates, such as what are called LORD trays or LORD reactors, which are described, for example, in DE-A1-10120801.

Preferred trays are tunnel-cap trays, bubble-cap trays, Streuber trays and modified bubble-cap trays (LORD trays).

In a further embodiment, the separating internals may, however, also be present as a structured packing, for example as a sheet metal packing, such as Mellapak 250 Y or Montz Pak, B1-250 type, or as a structured ceramic packing or as a random packing, for example composed of Pall rings, IMTP rings (from Koch-Glitsch), Raschig Superrings, etc. Structured or random packings may be arranged in one bed or preferably in a plurality of beds.

The reaction output from the first stage is preferably supplied in a spatial region between 50% and 100% of the theoretical plates of the stripping column (counted from the bottom), more preferably in a spatial region between 90% and 100% of the theoretical plates of the stripping column, especially at the uppermost tray. The optimal feed point can be determined by the person skilled in the art as a function of the composition of the reaction output from the first stage using the customary calculation tools.

The number of theoretical plates is generally in the range from 2 to 100, preferably 2 to 80, more preferably 20 to 70 and most preferably 25 to 60.

The residence time in the column is preferably 15 minutes to 300 minutes, more preferably 30 minutes to 240 minutes and most preferably 60 minutes to 240 minutes.

The top pressure is more preferably 500 to 3000 mbar, more preferably 800 to 2000 mbar and most preferably 1000 to 1500 mbar.

In the column bottom or at the point at which the stripping gas is supplied, preference is given to establishing a temperature above the evaporation temperature of the tertiary amine used, such that other low boilers and a portion of the water are converted completely or very substantially completely to the gas phase.

According to the invention a temperature which does not exceed 90° C., preferably 80° C. and more preferably 75° C. is established.

The temperature in the column bottom is preferably 50 to 90° C., more preferably 55 to 80° C. and most preferably 60 to 75° C.

For example, a column top pressure of 1013 mbar, preferably a column bottom temperature of 70° C., can be established.

Stripping gases are gases which are predominantly inert under the reaction conditions present and do not react with the substances present in the reaction mixture. The stripping gases used may be inert gases such as nitrogen, or noble gases, especially helium, neon, argon or xenon. Preference is given to using nitrogen. However, it is also possible here to use hydrogen. The inert gases used may also be mixtures of the aforementioned gases.

The stripping gas is preferably introduced into the lower region of the distillation column and hence conducted in countercurrent to the liquid stream.

The introduction can be effected into the bottom of the column, for example by means of a distributor ring or of a nozzle, but it can also be effected into the lower region of the distillation column, preferably in a spatial region up to 30%, preferably up to 20% and more preferably up to 10% of the theoretical plates of the distillation column (counted from the bottom). The stripping gas introduced is generally mixed thoroughly with the liquid flowing in the opposite direction by the internals present in the column.

In a preferred embodiment of the stripping column, the stripping gas used may be the hydrogenation offgas from the NPG process, which comprises predominantly hydrogen. In a particularly preferred embodiment, this hydrogenation gas, before being introduced into the stripping column, can be purified to free it of impurities, especially tertiary amine, by offgas scrubbing.

The flow rate of inert gas supplied is preferably 0.001 to 1, more preferably 0.005 to 0.1 and most preferably 0.01 to 0.05 m³/h of inert gas per kg/h of feed.

In the upper region of the column, generally unconverted isobutyraldehyde, formaldehyde, methanol and possibly water are drawn off as a gaseous stream and condensed in a condenser, and a portion of the condensate stream is preferably recycled to the column as a return stream. Optionally, a portion of the isobutyraldehyde can be recycled into the first reaction stage (aldol reaction).

The return stream at the top of the column is generally adjusted such that the predominant amount of HPA, IBA and water is retained in column, such that HPA is obtained virtually completely as the bottom product. The condensate obtained in the condenser is recycled into the top of the distillation column preferably to an extent of less than 50%, more preferably to an extent of less than 25%.

In the lower region of the stripping column, an output comprising preferably 40 to 80% by weight of HPA and more preferably 60 to 70% by weight of HPA is generally obtained.

In a preferred embodiment, the output from the stripping column has the following composition:

HPA: 40 to 80% by weight;

water: 10 to 50% by weight;

remainder: other organic compounds.

In a further preferred embodiment, the output from the stripping column has the following composition:

HPA: 60 to 70% by weight;

water: 15 to 40% by weight;

remainder: other organic compounds.

In a preferred embodiment, the pH of the output from the second reaction stage is 5 to 9, more preferably 6 to 8.

The isobutyraldehyde content is preferably less than 2% by weight, more preferably less than 1% by weight, even more preferably less than 0.5% by weight and especially preferably less than 0.1% by weight.

The trialkylamine content is preferably less than 2% by weight, more preferably less than 1% by weight and most preferably less than 0.5% by weight.

The formaldehyde content is preferably less than 8% by weight, more preferably less than 5% by weight and most preferably less than 2.5% by weight.

The content of ester of hydroxypivalic acid and NPG (=HPN) is preferably less than 2% by weight, more preferably less than 1% by weight and most preferably less than 0.5% by weight.

By means of the process according to the invention, it is possible to obtain an HPA-comprising output which can be used directly in the downstream hydrogenation stage (3rd reaction stage) for preparation of neopentyl glycol (NPG). In a preferred embodiment, further workup steps after the second reaction stage and before the third reaction stage are therefore not required.

By means of the process according to the invention, it is additionally possible to obtain an output which, without further addition of pH regulators, can be used directly in a further reaction stage in which the output from stage 2 is hydrogenated with hydrogen in the presence of a hydrogenation catalyst (3rd reaction stage=hydrogenation).

However, it is also possible to mix the output from the second reaction stage with pH regulators, for example tertiary amines, inorganic base, inorganic acid or organic acid, in order to regulate the pH until the hydrogenation output (output from the third reaction stage) has a pH of 7 to 9. It is also possible to feed the hydrogenation feed and pH regulators into the reactor separately and to mix them therein. The tertiary amines used may be the aforementioned tertiary amines, especially TMA.

In the hydrogenation (3rd reaction stage), preference is given to using catalysts which comprise at least one metal of transition groups 8 to 12 of the Periodic Table of the Elements, such as Fe, Ru, Os, Co, Rh, Ir, Ni, Pd, Pt, Cu, Ag, An, Zn, Cd, Hg, preferably Fe, Co, Ni, Cu, Ru, Pd, Pt, more preferably Cu, preferably on a support material.

The support material used is preferably a support material composed of the oxides of titanium, of zirconium, of hafnium, of silicon and/or of aluminum.

The usable catalysts can be prepared by processes known from the prior art for preparing such supported catalysts. Preference may also be given to using supported catalysts which comprise copper on an aluminum oxide- or titanium dioxide-containing support material in the presence or absence of one or more of the elements magnesium, barium, zinc or chromium. Such catalysts and preparation thereof are known from WO 99/44974.

In addition, supported copper catalysts as described, for example, in WO 95/32171 and the catalysts disclosed in EP-A 44 444 and DE 19 57 591 are suitable for the hydrogenation.

The hydrogenation can be performed batchwise or continuously, for example in a reactor tube filled with a catalyst bed, in which the reaction solution is passed over the catalyst bed, for example in trickle or liquid phase mode, as described in DE-A 19 41 633 or DE-A 20 40 501. It may be advantageous to recycle a substream of the reaction output, optionally with cooling, and to pass it through the fixed catalyst bed again. It may equally be advantageous to perform the hydrogenation in a plurality of reactors connected in series, for example in 2 to 4 reactors, in which case the hydrogenation reaction in the individual reactors upstream of the last reactor is performed only up to a partial conversion of, for example, 50 to 98%, and only in the last reactor is the hydrogenation completed. It may be appropriate to cool the hydrogenation output from the preceding reactor before its entry into the next reactor, for example by means of cooling apparatus or by injecting cold gases, such as hydrogen or nitrogen, or introducing a substream of cold reaction solution.

The hydrogenation temperature is generally between 50 and 180° C., preferably 90 and 140° C. The hydrogenation pressure employed is generally 10 to 250 bar, preferably 20 to 120 bar.

The reaction output from the third reaction stage (hydrogenation) comprises an NPG-containing mixture which preferably has the following composition:

20 to 90% by weight of NPG,

0 to 5% by weight of methanol,

0 to 5% by weight of tertiary amine,

0 to 5% by weight of organic secondary compounds,

0.01 to 5% by weight of the adduct of tertiary amine and formic acid (amine formate), remainder water.

The aqueous NPG mixture more preferably has the following composition:

50 to 80% by weight of NPG,

0.1 to 3% by weight of methanol,

0.01 to 5% by weight of tertiary amine,

0 to 5% by weight of organic secondary compounds,

0.01 to 5% by weight of the adduct of tertiary amine and formic acid (amine formate), remainder water.

The organic secondary compound present may, for example, be isobutanol.

The aqueous NPG mixture is preferably purified by removing low boilers from NPG. The low boilers are more preferably removed from the aqueous NPG mixture by distillation.

The distillation is preferably performed in such a way that low boilers, such as water, isobutanol, methanol and tertiary amine, are removed under reduced pressure via the top, especially when the amine used has a lower boiling point than the NPG formed, as is the case for TMA, TEA and TPA.

When a tertiary amine having a higher boiling point than the NPG formed is used, the tertiary amine is removed together with the NPG formed at the bottom and enriched in the column bottom in a downstream distillation stage, while NPG is drawn off as the top product.

Typically, a portion of the amine formates reacts during the distillation in the column bottom or in the stripping section of the column with NPG to form the free amines and the formates of NPG. This preferably forms the monoester of formic acid and NPG, which is referred to in the context of this disclosure as NPG formate.

The amines released by the transesterification reaction are generally removed in the distillation together with the other low boilers at the top of the column.

The distillation should therefore be regulated such that the concentration of the NPG formates formed in the bottom output is kept low and the target product, the NPG, is of maximum purity. This is preferably done by selecting, in the distillation, a bottom temperature above the evaporation temperature of the NPG formate, such that the NPG formates are completely or very substantially completely converted to the gas phase by evaporation.

The improvement in the yield and in the product quality brought about by this measure is probably attributable to the fact that the polymethylol formates typically have higher boiling points than the other low boilers, and the polymethylol formates are therefore generally precipitated in the rectifying section of the columns at an appropriate reflux ratio. The NPG formates precipitated in the rectifying section can hydrolyze with water to reform formic acid and NPG. The formic acid is typically removed at the top of the column, while NPG can generally be discharged from the column bottom.

In a preferred embodiment, the distillation is therefore preferably carried out as follows:

The condenser is generally operated at a temperature at which the predominant portion of the low boilers is condensed at the corresponding top pressure.

In general, the operating temperature of the condenser is in the range from 0 to 80° C., preferably 20 to 50° C.

The cooling medium used here may preferably be very cold water (e.g. about 5° C.) or a coolant mixture (e.g. glycol-water at, for example, −20° C.).

The top pressure is more preferably 0.001 to 0.9 bar, more preferably 0.01 to 0.5 bar.

On the industrial scale, the vacuum is typically obtained by means of a steam ejector. In the column bottom, preference is given to establishing a temperature which is above the evaporation temperature of the NPG formate, such that the NPG formate is converted completely or very substantially completely to the gas phase.

Particular preference is given to establishing a temperature which is 5% to 50% above the boiling temperature of the NPG formate and most preferably 10% to 20% above the boiling temperature of the NPG formate.

For example, in the case of preparation of NPG using TMA as the tertiary amine and a pressure at the top of the column of 175 mbar, a column bottom temperature of preferably 150 to 170° C., more preferably of 160 to 165° C., can be established.

The reflux at the top of the column is generally adjusted such that the predominant amount of the NPG formate is retained in the column.

The condensate obtained at the condenser is preferably recycled into the distillation column to an extent of more than 30% by weight, preferably to an extent of more than 60% by weight. The condensate is preferably recycled into the top of the column.

The energy required for the evaporation is typically introduced by means of an evaporator in the column bottom.

The evaporator is typically a natural circulation evaporator or forced circulation evaporator. However, it is also possible to use evaporators with a short residence time, falling film evaporators, helical tube evaporators, wiped film evaporators or a short path evaporator. The evaporator can be supplied with heat in a suitable manner, for example with 16 bar steam or heat carrier oil.

The distillation column preferably has internals for increasing the separating performance. The distillative internals may, for example, be present as a structured packing, for example as a sheet metal packing such as Mellapak 250 Y or Montz Pak, B1-250 type. It is also possible for a structured packing with relatively low or increased specific surface area to be present, or it is possible to use a fabric packing or a structured packing with another geometry, such as Mellapak 252Y. Advantages in the case of use of these distillative internals are the low pressure drop and the low specific liquid holdup compared to, for example, valve trays. The internals may be present in one or more sections.

The output from the hydrogenation is preferably fed in within a spatial region between ¼ and ¾ of the theoretical plates of the distillation column, more preferably in a spatial region between ⅓ and ⅔ of the theoretical plates of the distillation column. For example, the feed may be somewhat above the middle of the theoretical plates (ratio 3:4).

The number of theoretical plates is generally in the range from 5 to 30, preferably 10 to 20.

In the condenser, the condensate obtained is a mixture of low boilers which is supplied predominantly as a return stream to the column as described above. For example, the low boiler mixture may comprise amine, water and alcohols of the formula (III), such as isobutanol from isobutyraldehyde or n-butanol from n-butyraldehyde, and also methanol from formaldehyde.

The uncondensed residual vapors can be supplied in an energetically advantageous manner directly in gaseous form to combustion, or are supplied to a distillation column working close to ambient pressure. This downstream column serves for the further distillative separation of the condensate.

Preference is given to discharging an output comprising predominantly NPG from the bottom of the evaporator. Discharge from the circulation stream of the evaporator is also possible. The bottom output is referred to in the context of the present invention as “crude NPG”.

The crude NPG thus obtained comprises a small proportion of polymethylol formate. The proportion of polymethylol formate is preferably less than 1500 ppm by weight, preferably less than 1200 ppm by weight, more preferably less than 800 ppm by weight and especially preferably less than 600 ppm by weight.

The crude NPG further comprises NPG.

The crude NPG preferably has the following composition:

90 to 99% by weight of NPG (I),

0.01 to 5% by weight hydroxypivalic acid,

0 to 5% by weight of organic secondary compounds.

The crude NPG more preferably has the following composition:

95 to 99% by weight of NPG,

0.1 to 2% by weight of hydroxypivalic acid,

0 to 3% by weight of organic secondary compounds.

In order to remove the relatively high-boiling acidic components present in the bottoms, especially hydroxypivalic acid, with low loss of NPG, the bottoms evaporator used in the distillation is preferably at least one evaporator with short residence time, for example a falling film evaporator with residue discharge, a thin film evaporator or helical tube evaporator. In a particular embodiment, the bottom of the column may be configured as a tapering bottom, in order to further reduce the residence time in the column bottom.

The distillation of the crude NPG is preferably performed under the following conditions:

Advantageously, the condensate obtained in the condenser is recycled into the distillation column (return stream) to an extent of more than 30% by weight, more preferably to an extent of more than 50% by weight. The condensate is preferably recycled into the top of the column.

The condenser is preferably operated at a temperature in the range from 50 to 180° C., preferably 130 to 160° C.

The cooling medium used here may preferably as far as possible be water, which at the same time evaporates.

The top pressure is more preferably 0.001 to 0.9 bar, more preferably 0.01 to 0.5 bar and most preferably 0.02 to 0.4 bar.

The vacuum is typically generated on the industrial scale by means of a steam ejector.

The bottom temperature is generally selected such that NPG is converted to the gas phase, while hydroxypivalic acid remains in the column bottom.

Preference is given to establishing a bottom temperature which is 100 to 150%, preferably 105 to 140%, more preferably 110 to 130%, of the boiling temperature of the NPG.

For example, in the case of preparation of NPG using TMA as the tertiary amine and a pressure at the top of the column of 150 mbar, preference is given to establishing a column bottom temperature of 150 to 200° C., more preferably of 160 to 190° C.

The bottom of the distillation column is preferably connected to at least one evaporator with short residence time.

The bottom of the distillation column and the evaporator with short residence time together constitute, by definition, the evaporation stage.

According to the disclosure, the residence time of the evaporation stage is calculated by dividing the volume of the liquid holdup in the hot part of the column (V_{holdup}) by the sum of return stream and feed volume flow of the column (V_{holdup}/(feed stream+return stream)), the liquid holdup in the hot part of the column (V_{holdup}) being calculated from the volume of the holdup of the column bottom (V_{holdup, bottom}) plus the volume of the holdup of the evaporator (V_{holdup, evaporator}) (V_{holdup}=V_{holdup, bottom}+V_{holdup, evaporator}).

The residence time in the evaporation stage is advantageously less than 45 minutes, preferably less than 30 minutes, more preferably less than 15 minutes, especially preferably less than 10 minutes and most preferably less than 5 minutes.

In general, it is preferred to select the residence time in the evaporation stage such that a shorter residence time is correspondingly established at higher bottom temperatures.

At a bottom temperature which is in the range from 130 to 150% of the boiling temperature of the NPG, the residence time in the evaporation stage is preferably 5 minutes and less, more preferably 4 minutes and less, and most preferably 3 minutes and less.

At a bottom temperature which is within the range from 120 to 130% of the boiling temperature of the NPG, the residence time in the evaporation stage is preferably 30 minutes and less, more preferably 15 minutes and less and most preferably 10 minutes and less, and especially preferably 5 minutes and less.

At a bottom temperature which is within the range from 100 to 120% of the boiling temperature of the NPG, the residence time in the evaporation stage is preferably 45 minutes and less, more preferably 30 minutes and less and most preferably 15 minutes and less, and especially preferably 10 minutes and less.

In a further particular embodiment, the evaporator with short residence time is connected to at least one further evaporator with short residence time.

The bottom of the distillation column and the evaporator with short residence time, in this preferred embodiment, by definition, together constitute the first evaporation stage.

The further evaporator(s) with short residence time, by definition, form(s) the second or the (1+n)th (where n≧2) evaporation stage.

The evaporator with short residence time is preferably connected to one further evaporator with short residence time (two-stage configuration).

In this embodiment, the predominant portion of the energy needed for evaporation is usually introduced in the first evaporation stage. In the second evaporator stage, the higher temperature required for evaporation can then be achieved with a shorter residence time, such that the residence time in the second evaporation stage is shorter.

The first stage is preferably configured as a falling film evaporator or helical tube evaporator.

The second stage of this particular embodiment is preferably a falling film evaporator, helical tube evaporator or thin layer evaporator.

According to the disclosure, the residence time in the first evaporation stage is calculated by dividing the volume of the liquid holdup in the hot part of the column (V_{holdup}) by the sum of return stream and feed volume flow of the column (V_{holdup}/(feed stream+return stream)), the liquid holdup in the hot part of the column (V_{holdup}) being calculated from the volume of the holdup of the column bottom (V_{holdup, bottom}) plus the volume of the holdup of the evaporator (V_{holdup, evaporator}) (V_{holdup}=V_{holdup, bottom}+V_{holdup, evaporator}).

According to the disclosure, the residence time of the second evaporation stage is calculated by dividing the liquid holdup of the second evaporator by the feed stream of the second evaporator. According to the disclosure, the residence time of the (1+n)th evaporation stage is accordingly calculated by dividing the liquid holdup of the (1+n)th evaporator by the feed stream of the (1+n)th evaporator.

In this preferred embodiment, the bottom temperature in the first evaporation stage is advantageously above the evaporation temperature of the NPG.

The bottom temperature in the first evaporation stage is preferably 100 to 130%, more preferably 110 to 125%, above the boiling temperature of the NPG.

The temperature in the second evaporation stage is generally selected such that the NPG is converted virtually completely to the gas phase.

The temperature in the second evaporation stage is preferably 105 to 150%, more preferably 120 to 150%, especially preferably 130 to 140%, above the boiling temperature of the NPG. The residence time in the first evaporation stage is advantageously less than 45 minutes, preferably less than 30 minutes, more preferably less than 15 minutes, especially preferably less than 10 minutes and most preferably less than 5 minutes.

The residence time in the second evaporation stage is advantageously less than 30 minutes, preferably less than 15 minutes, more preferably less than 5 minutes, especially preferably less than 2 minutes and most preferably less than 1 minute.

In general, it is preferred to select the residence time of the evaporation stage such that a shorter residence time is established correspondingly at higher bottom temperatures.

As mentioned above, the evaporator with short residence time can be connected to more than one further evaporator with short residence time, for example to 2 or 3 evaporators, in which case the last of the evaporators in the chain constitutes the so-called last evaporation stage. The residence time and the temperatures in the last evaporation stage correspond to the residence times and temperatures of the second evaporation stage in the two-stage configuration.

In the preparation of NPG using TMA as the tertiary amine, in the first evaporation stage, a bottom temperature of 135 to 170° C., more preferably 150 to 160° C., can preferably be established at a residence time of less than 45 minutes, preferably less than 30 minutes. In the second evaporation stage, a temperature of 160 to 220° C., preferably 180 to 200° C., is preferably established at a residence time of less than 15 minutes, preferably less than 10 minutes and more preferably less than 5 minutes.

The distillation column preferably has internals for increasing the separating performance. The distillative internals may, for example, be present as a structured packing, for example as a sheet metal packing such as Mellapak 250 Y or Montz Pak, B1-250 type. It is also possible for a structured packing with relatively low or increased specific surface area to be present, or it is possible to use a fabric packing or a structured packing with another geometry such as Mellapak 252 Y. Advantages in the case of use of these distillative internals are the low pressure drop and the low specific liquid holdup compared to, for example, valve trays. The internals may be present in one or more sections.

The hydrogenation output is preferably fed in within a spatial region between ¼ and ¾ of the theoretical plates of the distillation column, more preferably within a spatial region between ⅓ and ⅔ of the theoretical plates of the distillation column. For example, the feed may be somewhat above the middle of the theoretical plates (ratio 3:4).

The number of theoretical plates is generally in the range from 5 to 30, preferably 10 to 20.

Under these conditions, in general, NPG is removed from the higher-boiling hydroxypivalic acid.

In the condenser, purified NPG is preferably obtained as the condensate.

The purity of the NPG is preferably at least 99.0% by weight, more preferably at least 99.2% by weight.

Preference is given to discharging, from the bottom of the evaporator, an output which comprises a predominantly higher-boiling compound, such as hydroxypivalic acid.

The bottoms can either be utilized thermally in an incineration or be fed to a downstream distillation column, by fractionating it into several fractions.

For example, the bottoms, in the case of preparation of NPG, can be fractionated into a low-boiling fraction, in particular containing hydroxypivalic acid, a medium-boiling fraction, in particular containing HPN (>97% HPN), and a high-boiling fraction (in particular esters of HPA and HPN).

The uncondensed residual vapors comprise generally, as well as leakage air and traces of water, predominantly NPG, and are advantageously recycled directly in gaseous form into distillation stage d).

NPG is used principally as a component for the synthesis of polyester resins, unsaturated polyester resins, lubricants and plasticizers.

The present invention accordingly also relates to a process for producing polyester resins, unsaturated polyester resins, lubricants or plasticizers, which comprises preparing NPG in accordance with the invention and using the NPG thus prepared for the production of polyester resins, unsaturated polyester resins, lubricants and plasticizers.

The advantages of the present invention are that it is possible by means of the process according to the invention to prepare HPA which can be introduced directly into a downstream hydrogenation stage in which HPA is converted to NPG.

The HPA prepared in accordance with the invention generally need not be purified any further before use in the downstream hydrogenation. The HPA prepared in accordance with the invention generally has a low content of HPN. There is preferably also no need to add any pH regulators to the hydrogenation feed in order to obtain a pH in the range from 7.0 to 9.0. The process according to the invention additionally enables the preparation of an HPA which leads to higher yields and NPG selectivities in a downstream hydrogenation stage. In addition, the number of formaldehyde equivalents used in the aldolization reaction can be reduced in order to save raw material costs. In addition, the content of unconverted isobutyraldehyde in the output from the second reaction stage of the aldolization is low.

### EXAMPLES

The examples which follow are based on simulation results which were achieved with the Chemasim™ software. The thermodynamic parameters used in the program for the reactants, products and by-products are based on published thermodynamic data or in-house measurements. The kinetic constants used for the main reaction were verified experimentally; the kinetics of the side reactions were estimated.

The specification and simulation of the specified apparatuses used were effected with the customary routines included in the software.

To optimize the simulation model, the simulated results were compared with experimental results, where available, and the simulation model was brought in line with the experimental results, such that a good agreement could be achieved between simulation and experimental data.

The examples which follow were calculated with the optimized simulation model.

### Comparative Example

### Simulation

Approx. 750 g/h of isobutyraldehyde (approx. >99.5 GC area% of IBA) reacted with approx. 700 g/h of formaldehyde (approx. 49% Fa, 1.5% methanol, remainder water) and 80 g/h of trimethylamine solution (50% TMA in water) in a two-stage stirred tank cascade.

Subsequently, the solution was freed of low boilers by distillation in a column.

The column is equipped with 1.5 m of fabric packing (specific surface area 500 m²/m³) in the rectifying section and 4 m of sheet metal packing (250 m²/m³). The aldolization output was supplied above the sheet metal packing; at the top of the column, a condenser with cooling water (approx. 10° C.) and a downstream phase separator was used. At the top, the distillate was supplied in gaseous form to the condenser. Approx. 255 g/h of liquid condensate were obtained. In the downstream phase separator, an aqueous phase of 95 g/h was removed and supplied fully to the column. In addition, 135 g/h from the phase separator was supplied to the first stirred tank. In order to maintain the regulation temperature in the column at 85° C., 25 g/h of organic phase were additionally supplied to the column. In the cold trap downstream of the condenser, approx. 1 g/h of liquid were obtained (approx. 80% IBA, approx. 20% TMA), which were likewise recycled.

The IBA removal was conducted at a top pressure of approx. 1 bar absolute. The evaporator used was a falling-film evaporator. A bottom temperature in the bottom of the column of 100° C. was established. The reflux rate to the column was regulated by means of the temperature in the middle of the fabric packing; a temperature of 85° C. was established.

From the column bottom, approx. 15 kg/h of liquid were drawn off by means of a pump. This liquid was supplied to the falling-film evaporator (consisting of an oil-heated stainless steel tube, length 2.5 m, internal diameter approx. 21 mm, wall thickness approx. 2 mm). Approx. 1.5 kg/h of product were drawn off from the bottom of the falling-film evaporator. The vapors and excess liquid were supplied to the column bottom. The bottom product discharged comprised approx. 70% by weight of HPA, approx. 1% by weight of HPN, 0.7% by weight of IBA, remainder water.

### Example 1

### Simulation

Approx. 750 g/h of isobutyraldehyde (approx. >99.5 GC area% of IBA) were reacted with approx. 700 g/h of formaldehyde (approx. 49% Fa, 1.5% methanol, remainder water) and 80 g/h of trimethylamine solution (50% TMA in water) in a two-stage stirred tank cascade.

Subsequently, the solution was freed of low boilers in a stripping column by addition of a stripping gas.

The column is equipped with 12 theoretical plates (plate efficiency approx. 30%, corresponds to about 40 bubble-cap trays). The aldolization output was supplied to the uppermost tray; at the top of the column, a condenser was operated with cold water (approx. 2° C.). The gaseous stream exiting at the top was supplied to the condenser. Approx. 60 g/h of liquid condensate were obtained. The condensate was supplied fully to the uppermost tray of the column.

The stripping column was operated at a top pressure of approx. 1 bar absolute. In the bottom, 60 g/h of stripping gas (nitrogen) were supplied. By means of trace heating, the bottom temperature was adjusted to a value of 65° C.

From the column bottom, approx. 1.5 kg/h of liquid were drawn off by means of a pump. The bottom product discharged comprised approx. 70% by weight of HPA, approx. 0.4% by weight of HPN, 0.7% by weight of IBA, remainder water.

1-12. (canceled)

13. A process for preparing hydroxypivalaldehyde (HPA), which comprises
in a first stage reacting isobutyraldehyde (IBA) with formaldehyde (FA) in the presence of a tertiary amine and
in a second stage introducing the reaction output obtained from the first stage into a stripping column, with introduction of a stripping gas into the stripping column.

14. The process according to claim 13, wherein the residence time in the stripping column is 60 to 240 minutes.

15. The process according to claim 13, wherein the stripping gas is an inert gas.

16. The process according to claim 15, wherein the flow rate of inert gas supplied is 0.005 to 0.1 m3/h of inert gas per kg/h of feed.

17. The process according to claim 13, wherein the reaction output from the first stage comprises 40 to 80% by weight of HPA.

18. The process according to claim 13, wherein the reaction output from the first stage has the following composition:
HPA: 40 to 80% by weight;
water: 10 to 50% by weight;
IBA: 0 to 20% by weight;
FA: 0 to 10% by weight;
tert. amine: 0 to 10% by weight.

19. The process according to claim 13, wherein the bottom temperature of the stripping column is 90° C. or less.

20. The process according to claim 13, wherein the number of theoretical plates in the stripping column is in the range from 2 to 80.

21. The process according to claim 13, wherein the stripping column is a tray column.

22. The process according to claim 13, wherein the stripping column has tunnel-cap trays, bubble-cap trays or Streuber trays.

23. A process for preparing neopentyl glycol, which comprises
in a first stage reacting isobutyraldehyde with formaldehyde in the presence of a tertiary amine;
in a second stage introducing the reaction output obtained from the first stage into a stripping column; and
in a third stage hydrogenating the output from the stripping column in the presence of a hydrogenation catalyst, with introduction of a stripping gas into the second stage stripping column

24. A process for producing polyester resins, unsaturated polyester resins, lubricants or plasticizers, which comprises preparing neopentyl glycol according to claim 23 and using the neopentyl glycol thus prepared for the production of polyester resins, unsaturated polyester resins, lubricants and plasticizers.
